(19) United States
(12) Patent Application Publication
(10) Pub. No.: US 2017/0133604 A1
(43) Pub. Date: May 11, 2017

(54) ORGANIC LIGHT EMITTING DEVICE AND DISPLAY DEVICE HAVING THE SAME

(71) Applicant: SAMSUNG DISPLAY CO., LTD., Yongin-si (KR)

(72) Inventors: Youngtak LEE, Suwon-si (KR); Jahyun IM, Yongin-si (KR); Hyundo JIN, Yongin-si (KR)

(21) Appl. No.: 15/194,611

(22) Filed: Jun. 28, 2016

(30) Foreign Application Priority Data
Nov. 6, 2015 (KR) 10-2015-0155937

(51) Int. Cl. H01L 51/00; C07D 493/04

(52) U.S. Cl. CPC H01L 51/0073; C07D 493/04; H01L 51/0058; H01L 51/0085; H01L 2251/558; H01L 2251/301; H01L 27/3244

(57) ABSTRACT

An organic light emitting device and a display device, the organic light emitting device including an anode; a hole transport region on the anode; an emission layer on the hole transport region; a buffer layer on the emission layer; an electron transport region on the buffer layer; and a cathode on the electron transport region, wherein the buffer layer includes a buffer compound represented by the following Formula 1:

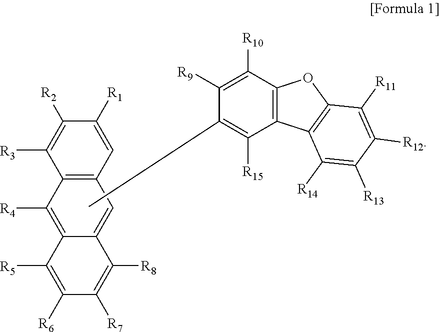

### CROSS-REFERENCE TO RELATED APPLICATION

Korean Patent Application No. 10-2015-0155937, filed on Nov. 6, 2015, in the Korean Intellectual Property Office, and entitled: “Organic Light Emitting Device and Display Device Having the Same,” is incorporated by reference herein in its entirety.

### BACKGROUND

1. Field

Embodiments relate to an organic light emitting device and a display device having the same.

2. Description of the Related Art

Flat panel display devices may be mainly classified as a light emitting type and a light receiving type. The light emitting type may include a flat cathode ray tube, a plasma display panel, and an organic light emitting display (OLED). The OLED is a self-luminescent display and has wide viewing angles, good contrast, and rapid response times.

Accordingly, the OLED may be applied to display devices for mobile devices such as digital cameras, video cameras, camcorders, portable information terminals, smart phones, ultra slim laptops, tablet personal computers, and flexible display devices, large-sized electronic products such as ultra slim televisions, or large-sized electric products, and receives much attention.

The OLED may reproduce colors on the basis of emitting light via the recombination of holes and electrons injected from an anode and a cathode in an emission layer, and light is emitted by the transition of excitons obtained by the recombination of the injected holes and electrons from an excited state to a ground state.

### SUMMARY

Embodiments are directed to an organic light emitting device and a display device having the same.

The embodiments may be realized by providing an organic light emitting device including an anode; a hole transport region on the anode; an emission layer on the hole transport region; a buffer layer on the emission layer; an electron transport region on the buffer layer; and a cathode on the electron transport region, wherein the buffer layer includes a buffer compound represented by the following Formula 1:

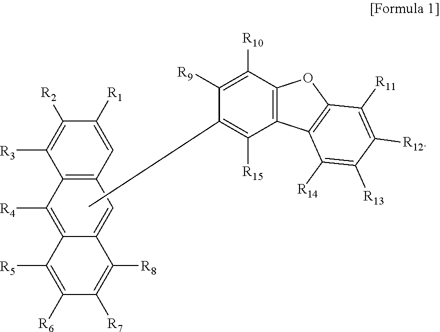

wherein, in Formula 1, R₁ to R₁₈ are each independently hydrogen, deuterium, a substituted or unsubstituted aromatic group, or a substituted or unsubstituted heteroaromatic group, adjacent ones of R₁ to R₁₈ being separate or fused to form substituted or unsubstituted condensed aromatic groups or substituted or unsubstituted condensed heteroaromatic groups.

The buffer compound may be represented by one of the following Formula 2, Formula 3, or Formula 4:

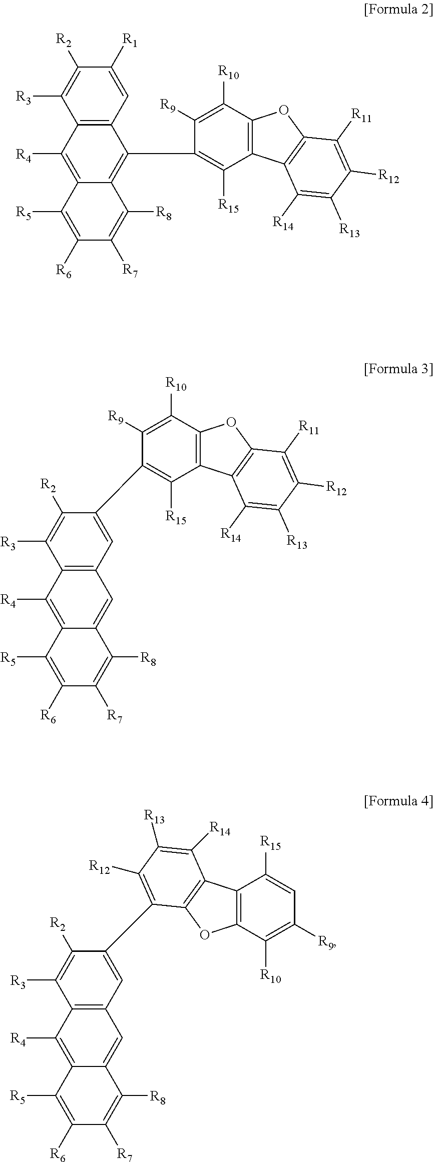

wherein, in Formulae 2, 3, and 4, R₁ to R₁₈ are defined the same as R₁ to R₁₈ of Formula 1.

The buffer compound may be one of the following compounds:

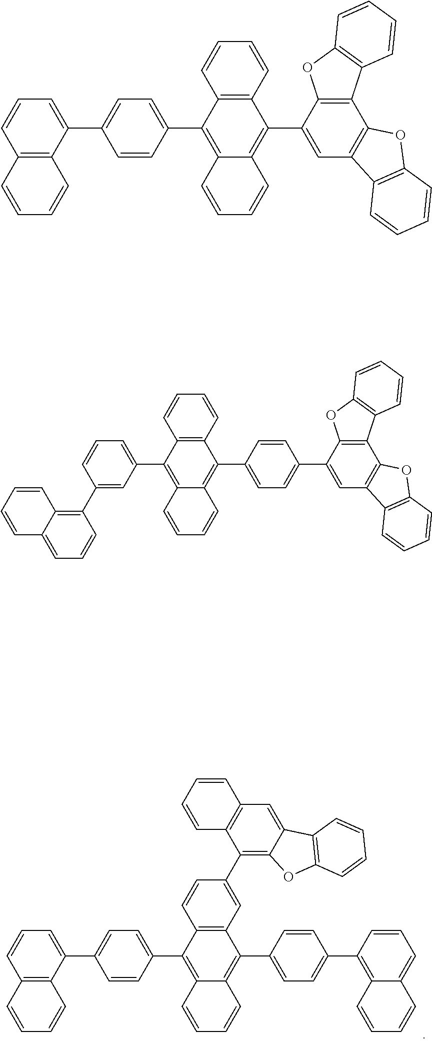 [SMILES: c1ccc2c(-c3ccc(-c4c5ccccc5c(-c5cc6c7ccccc7oc6c6c5oc5ccccc56)c5ccccc45)cc3)cccc2c1 ; c1cc(-c2cccc3ccccc23)cc(-c2c3ccccc3c(-c3ccc(-c4cc5c6ccccc6oc5c5c4oc4ccccc45)cc3)c3ccccc23)c1 ; c1ccc2c(-c3ccc(-c4c5ccccc5c(-c5ccc(-c6cccc7ccccc67)cc5)c5cc(-c6c7ccccc7cc7c6oc6ccccc67)ccc45)cc3)cccc2c1]

A thickness of the buffer layer may be about 10 Å to about 150 Å.

The buffer layer may further include a dopant.

The dopant may include Ir, Pt, Os, Au, Cu, Re, Ru, or an anthracene group-containing compound.

A thickness of the buffer layer may be about 10 Å to about 400 Å.

The hole transport region may include a hole injection layer; and a hole transport layer on the hole injection layer.

The electron transport region may include an electron transport layer; and an electron injection layer on the electron transport layer.

The embodiments may be realized by providing a display device including a plurality of pixels, wherein at least one of the pixels includes an anode; a hole transport region on the anode; an emission layer on the hole transport region; a buffer layer on the emission layer; an electron transport region on the buffer layer; and a cathode on the electron transport region, wherein the buffer layer includes a buffer compound represented by the following Formula 1:

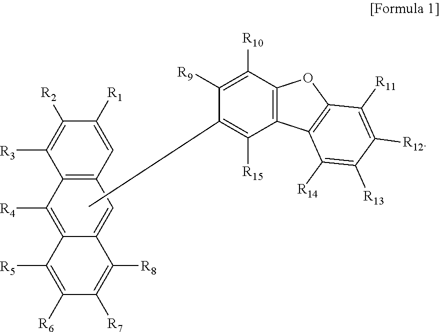

wherein, in Formula 1, R₁ to R₁₈ are each independently hydrogen, deuterium, a substituted or unsubstituted aromatic group, or a substituted or unsubstituted heteroaromatic group, adjacent ones of R₁ to R₁₈ being separate or fused to form substituted or unsubstituted condensed aromatic groups or substituted or unsubstituted condensed heteroaromatic groups.

The buffer compound may be represented by one of the following Formula 2, Formula 3, or Formula 4:

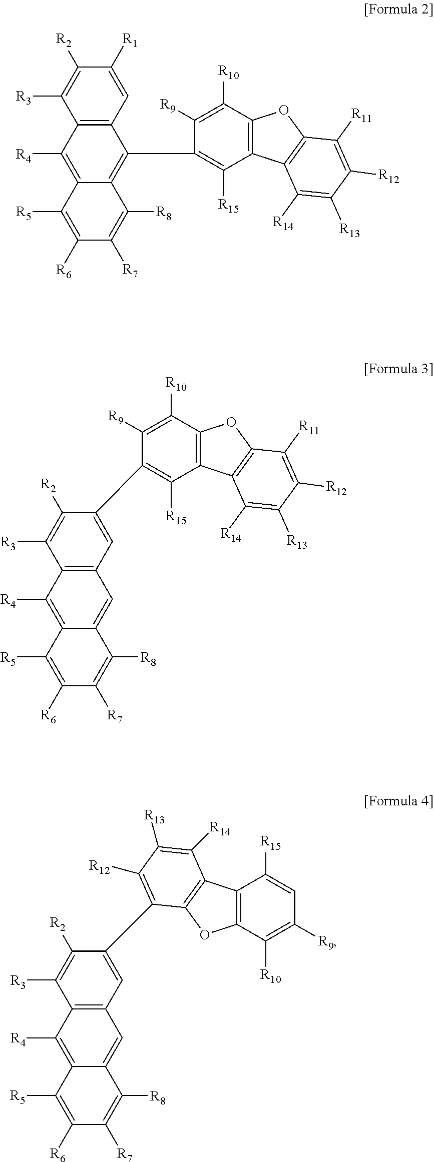

wherein, in Formulae 2, 3, and 4, R₁ to R₁₈ are defined the same as R₁ to R₁₈ of Formula 1

The buffer compound may be one of the following compounds:

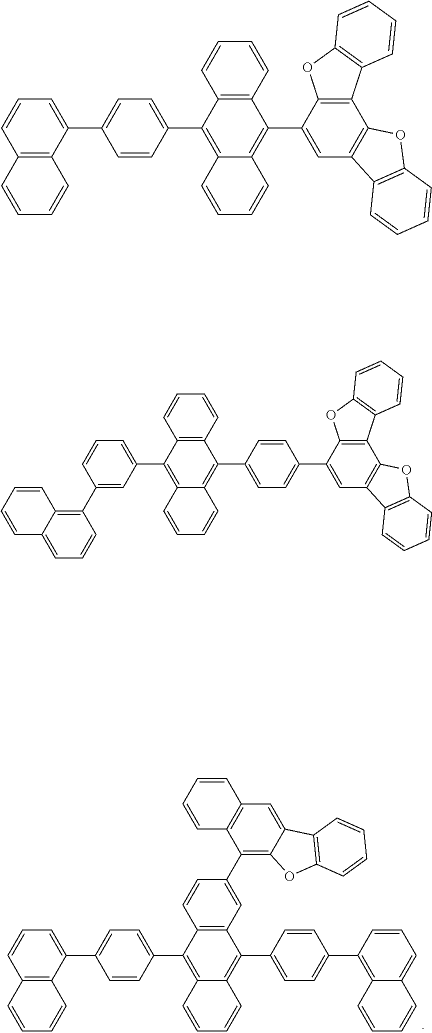 [SMILES: c1ccc2c(-c3ccc(-c4c5ccccc5c(-c5cc6c7ccccc7oc6c6c5oc5ccccc56)c5ccccc45)cc3)cccc2c1 ; c1cc(-c2cccc3ccccc23)cc(-c2c3ccccc3c(-c3ccc(-c4cc5c6ccccc6oc5c5c4oc4ccccc45)cc3)c3ccccc23)c1 ; c1ccc2c(-c3ccc(-c4c5ccccc5c(-c5ccc(-c6cccc7ccccc67)cc5)c5cc(-c6c7ccccc7cc7c6oc6ccccc67)ccc45)cc3)cccc2c1]

A thickness of the buffer layer may be about 10 Å to about 150 Å.

The buffer layer may further include a dopant.

The dopant may include Ir, Pt, Os, Au, Cu, Re, Ru, or an anthracene group-containing compound.

A thickness of the buffer layer may be about 10 Å to about 400 Å.

### BRIEF DESCRIPTION OF THE DRAWINGS

Features will be apparent to those of skill in the art by describing in detail exemplary embodiments with reference to the attached drawings in which:

FIG. 1 illustrates a schematic cross-sectional view of an organic light emitting device according to an embodiment;

FIG. 2 illustrates a schematic cross-sectional view of an organic light emitting device according to an embodiment;

FIG. 3 illustrates a schematic perspective view of a display device according to an embodiment;

FIG. 4 illustrates a circuit diagram of one pixel included in a display device according to an embodiment;

FIG. 5 illustrates a plan view of one pixel included in a display device according to an embodiment;

FIG. 6 illustrates a schematic cross-sectional view corresponding to line I-I′ in FIG. 5;

FIG. 7 illustrates a graph showing luminous efficiency relative to grey levels in Examples 1, 2, and 3, and the Comparative Example;

FIG. 8 illustrates a graph showing luminous efficiency relative to grey levels in Examples 1, 2, and 3, and the Comparative Example;

FIG. 9 illustrates a graph showing luminous efficiency relative to grey levels in Examples 4, and 5, and the Comparative Example; and

FIG. 10 illustrates a graph showing luminous efficiency relative to grey levels in Examples 4, 5, and 6, and the Comparative Example.

### DETAILED DESCRIPTION

Example embodiments will now be described more fully hereinafter with reference to the accompanying drawings; however, they may be embodied in different forms and should not be construed as limited to the embodiments set forth herein. Rather, these embodiments are provided so that this disclosure will be thorough and complete, and will fully convey exemplary implementations to those skilled in the art.

In the drawing figures, the dimensions of layers and regions may be exaggerated for clarity of illustration. In addition, it will also be understood that when a layer is referred to as being “between” two layers, it can be the only layer between the two layers, or one or more intervening layers may also be present. Like reference numerals refer to like elements throughout.

It will be understood that, although the terms first, second, etc. may be used herein to describe various elements, these elements should not be limited by these terms. These terms are only used to distinguish one element from another element. Thus, a first element could be termed a second element without departing from the teachings herein. Similarly, a second element could be termed a first element. As used herein, the singular forms are intended to include the plural forms as well, unless the context clearly indicates otherwise.

It will be further understood that the terms “comprises” “includes,” “including,” and/or “comprising,” when used in this specification, specify the presence of stated features, numerals, steps, operations, elements, parts, or the combination thereof, but do not preclude the presence or addition of one or more other features, numerals, steps, operations, elements, parts, or the combination thereof. It will also be understood that when a layer, a film, a region, a plate, etc. is referred to as being ‘on’ another part, it can be directly on the other part, or intervening layers may also be present. On the contrary, it will be understood that when a layer, a film, a region, a plate, etc. is referred to as being ‘under’ another part, it can be directly under, and one or more intervening layers may also be present.

Hereinafter, an organic light emitting device according to an embodiment will be explained.

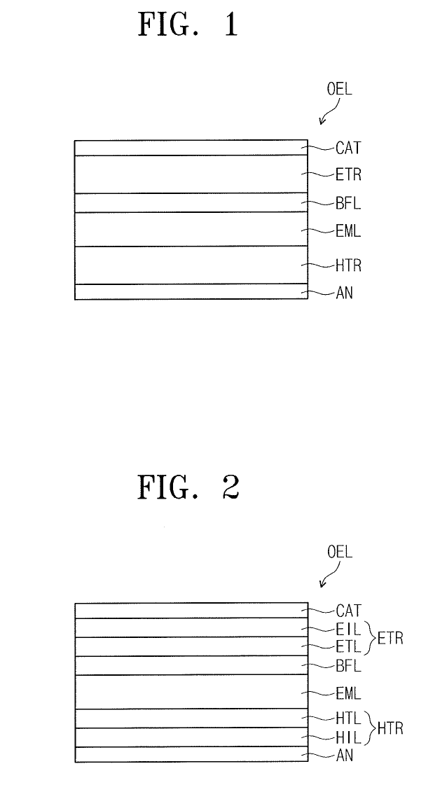

FIG. 1 illustrates a schematic cross-sectional view of an organic light emitting device according to an embodiment. FIG. 2 illustrates a schematic cross-sectional view of an organic light emitting device according to an embodiment.

Referring to FIGS. 1 and 2, an organic light emitting device OEL according to an embodiment may include, e.g., an anode AN, a hole transport region HTR, an emission layer EML, a buffer layer BFL, an electron transport region ETR, and a cathode CAT. For example, the buffer layer BFL may be between the emission layer EML and the electron transport region ETR.

The anode AN has conductivity. The anode AN may be a pixel electrode or an anode. The anode AN may be a transmissive electrode, a transflective electrode, or a reflective electrode. When the anode AN is the transmissive electrode, the anode AN may be formed using a transparent metal oxide such as indium tin oxide (ITO), indium zinc oxide (IZO), zinc oxide (ZnO), or indium tin zinc oxide (ITZO). When the anode AN is a transflective electrode or a reflective electrode, the anode AN may include Ag, Mg, Cu, Al, Pt, Pd, Au, Ni, Nd, Ir, Cr, Li, Ca, LiF/Ca, LiF/Al, Mo, Ti, a compound thereof, or a mixture thereof (for example, a mixture of Ag and Mg). Also, the anode AN may include a plurality of layers including a reflective layer or a transflective layer formed using the above materials, and a transmissive layer formed using ITO, IZO, ZnO, or ITZO.

The hole transport region HTR may be provided on the anode AN. The hole transport region HTR may include at least one of a hole injection layer HIL, a hole transport layer HTL, a hole buffer layer, or an electron blocking layer. The thickness of the hole transport layer HTR may be, for example, from about 1,000 Å to about 1,500 Å.

The hole transport region HTR may have a single layer formed using a single material, a single layer formed using a plurality of different materials, or a multilayer structure including a plurality of layers formed using a plurality of different materials.

For example, the hole transport region HTR may have the structure of a single layer such as a hole injection layer HIL, and a hole transport layer HTL, and may have a structure of a single layer formed using a hole injection material and a hole transport material. In addition, the hole transport region HTR may have a structure of a single layer formed using a plurality of different materials, or a structure laminated from the anode AN of hole injection layer HIL/hole transport layer HTL, hole injection layer HIL/hole transport layer HTL/hole buffer layer, hole injection layer HIL/hole buffer layer, hole transport layer HTL/hole buffer layer, or hole injection layer HIL/hole transport layer HTL/electron blocking layer.

The hole transport region HTR may be formed using various methods such as a vacuum deposition method, a spin coating method, a cast method, a Langmir-Blodgett (LB) method, an inkjet printing method, a laser printing method, and a laser induced thermal imaging (LITI) method.

When the hole transport region HTR includes the hole injection layer HIL, the hole transport region HTR may include a phthalocyanine compound such as copper phthalocyanine, N,N′-diphenyl-N,N′-bis-[4-(phenyl-m-tolyl-amino)-phenyl]-biphenyl-4,4′-diamine (DNTPD), 4,4′,4″-tris(3-methylphenylphenylamino)triphenylamine (m-MTDATA), 4,4′,4″-tris(N,N-diphenylamino)triphenylamine (TDATA), 4,4′,4″-tris{N-(2-naphthyl)-N-phenylamino}-triphenylamine (2-TNATA), poly(3,4-ethylenedioxythiophene)/poly(4-styrenesulfonate) (PEDOT/PSS), polyaniline/dodecylbenzenesulfonic acid (PANI/DBSA), polyaniline/camphor sulfonic acid (PANI/CSA), polyaniline/poly(4-styrenesulfonate) (PANI/PSS), etc.

When the hole transport region HTR includes the hole transport layer HTL, the hole transport region HTR may include a carbazole derivative such as N-phenylcarbazole and polyvinyl carbazole, a fluorine-based derivative, N,N′-bis(3-methylphenyl)-N,N′-diphenyl-[1,1-biphenyl]-4,4′-diamine (TPD), a triphenylamine-based derivative such as 4,4′,4″-tris(N-carbazolyl)triphenylamine (TCTA), N,N′-di(1-naphthyl)-N,N′-diphenylbenzidine (NPB), 4,4′-cyclohexylidene bis[N,N-bis(4-methylphenyl)benzeneamine] (TAPC), etc.

The thickness of the hole transport region HTR may be from about 100 Å to about 10,000 Å, for example, from about 100 Å to about 1,000 Å. When the hole transport region HTR includes both the hole injection layer HIL and the hole transport layer HTL, the thickness of the hole injection layer HIL may be from about 100 Å to about 10,000 Å, for example, from about 100 Å to about 1,000 Å, and the thickness of the hole transport layer HTL may be from about 50 Å to about 2,000 Å, for example, from about 100 Å to about 1,500 Å. When the thicknesses of the hole transport region HTR, the hole injection layer HIL, and the hole transport layer HTL satisfy the above-described ranges, satisfactory hole transport properties may be obtained without a substantial increase of a driving voltage.

The hole transport region HTR may further include a charge generating material other than the above-described materials to improve conductivity. The charge generating material may be dispersed in the hole transport region HTR uniformly or non-uniformly. The charge generating material may be, for example, a p-dopant. The p-dopant may be one of a quinone derivative, a metal oxide, or a cyano group-containing compound. Examples of the p-dopant may include a quinone derivative such as tetracyanoquinodimethane (TCNQ), and 2,3,5,6-tetrafluoro-tetracyanoquinodimethane (F4-TCNQ), a metal oxide such as tungsten oxide, and molybdenum oxide.

As described above, the hole transport region HTR may further include one of the hole buffer layer and the electron blocking layer other than the hole injection layer HIL and the hole transport layer HTL. The hole buffer layer may compensate an optical resonance distance according to the wavelength of light emitted from the emission layer EML and increase light emission efficiency. Materials included in the hole transport region HTR may be used as materials included in the hole buffer layer. The electron blocking layer is a layer for reducing and/or preventing electron injection from the electron transport region ETR to the hole transport region HTR.

The emission layer EML may be provided on the hole transport region HTR. The thickness of the emission layer EML may be from about 100 Å to about 300 Å. The emission layer EML may have a single layer formed using a single material, a single layer formed using a plurality of different materials, or a multilayer structure having a plurality of layers formed using a plurality of different materials.

The emission layer EML may emit one of red light, green light, blue light, white light, yellow light, or cyan light. The emission layer EML may include a phosphorescent material or a fluorescent material. In addition, the emission layer EML may include a host and/or a dopant.

The host may include, for example, tris(8-hydroxyquinolino)aluminum (Alq3), 4,4′-bis(N-carbazolyl)-1,1′-biphenyl (CBP), poly(n-vinylcarbazole) (PVK), 9,10-di(naphthaline-2-yl)anthracene (ADN), 4,4′,4″-tris(carbazole-9-yl)-triphenylamine (TCTA), 1,3,5-tris(N-phenylbenzimidazole-2-yl)benzene (TPBi), 3-tert-butyl-9,10-di(naphth-2-yl)anthracene (TBADN), distyrylarylene (DSA), 4,4′-bis(9-carbazolyl)-2,2′-dimethyl-biphenyl (CDBP), 2-methyl-9,10-bis(naphthalen-2-yl)anthracene (MADN), or the like.

The dopant may include, for example, styryl derivatives (for example, 1,4-bis[2-(3-N-ethylcarbazolyl)vinyl]benzene (BCzVB), 4-(di-p-tolylamino)-4′-[di-p-tolylamino]styryl]stilbene (DPAVB), N-(4-((E)-2-(6-((E)-4-(diphenylamino)styryl)naphthalen-2-yl)vinyl)phenyl)-N-phenylbenzenamine (N-BDAVBi)), perylene and the derivatives thereof (for example, 2,5,8,11-tetra-t-butylperylene (TBP)), pyrene and the derivatives thereof (for example, 1,1-dipyrene, 1,4-dipyrenylbenzene, 1,4-bis(N,N-diphenylamino)pyrene), or the like.

When the emission layer EML emits red light, the emission layer EML may include a phosphorescent material including, for example, tris(dibenzoylmethanato)phenanthroline europium (PBD:Eu(DBM)₃(Phen)), or perylene. When the emission layer EML emits red light, the dopant included in the emission layer EML may be selected from a metal complex or an organometallic complex such as bis(1-phenylisoquinoline)acetylacetonate iridium (PIQIr(acac)), bis(1-phenylquinoline)acetylacetonate iridium (PQIr(acac), tris(1-phenylquinoline)iridium (PQIr), and octaethylporphyrin platinum (PtOEP).

When the emission layer EML emits green light, the emission layer EML may include a phosphorescent material including, for example, tris(8-hydroxyquinolino)aluminum (Alq3). When the emission layer EML emits green light, the dopant included in the emission layer EML may be selected from a metal complex or an organometallic complex such as fac-tris(2-phenylpyridine)iridium (Ir(ppy)₃).

When the emission layer EML emits blue light, the emission layer EML may further include a phosphorescent material including, for example, spiro-DPVBi, spiro-6P, distyryl-benzene (DSB), distyryl-arylene (DSA), a polyfluorene (PFO)-based polymer, or a poly(p-phenylene vinylene) (PPV)-based polymer. When the emission layer EML emits blue light, the dopant included in the emission layer EML may be selected from a metal complex or an organometallic complex such as (4,6-F₂ppy)₂Irpic.

The buffer layer BFL may be provided on the emission layer EML. The buffer layer may include, e.g., a buffer compound represented by the following Formula 1.

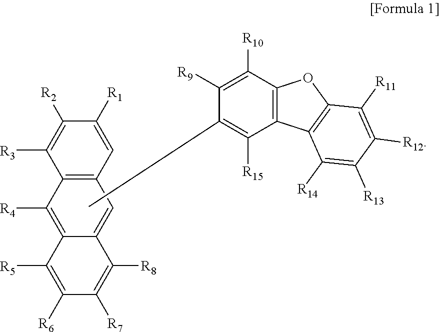

In Formula 1, R₁ to R₁₈ may each independently be or include, e.g., hydrogen, deuterium, a substituted or unsubstituted aromatic group, or a substituted or unsubstituted heteroaromatic group. In an implementation, adjacent ones of R₁ to R₁₈ may be separate or may be fused to form substituted or unsubstituted condensed aromatic groups or substituted or unsubstituted condensed heteroaromatic groups.

In the description, the terms “substituted or unsubstituted” corresponds to substituted or unsubstituted with at least one substituent selected from deuterium, a halogen group, a nitrile group, a nitro group, an amino group, a phosphine oxide group, an alkoxy group, an aryloxy group, an alkylthioxy group, an arylthioxy group, an alkylsulfoxy group, an arylsulfoxy group, a silyl group, a boron group, an alkyl group, a cycloalkyl group, an alkenyl group, an aryl group, an aralkyl group, an aralkenyl group, an alkylaryl group, an alkylamine group, a heteroarylamine group, an arylamine group, and a heterocyclic group, or corresponds to substituted or unsubstituted with a substituent obtained by connecting at least two substituents of the above-described substituents. For example, the substituent obtained by connecting at least two substituents may be a biphenyl group. For example, the biphenyl group may be an aryl group or may be interpreted as a substituent obtained by connecting two phenyl groups.

In an implementation, in Formula 1, R₁ to R₁₈ may each independently be selected from, e.g., deuterium, a halogen group, a nitrile group, a nitro group, an amino group, a phosphine oxide group, an alkoxy group, an aryloxy group, an alkylthioxy group, an arylthioxy group, an alkylsulfoxy group, an arylsulfoxy group, a silyl group, a boron group, an alkyl group, a cycloalkyl group, an alkenyl group, an aryl group, an aralkyl group, an aralkenyl group, an alkylaryl group, an alkylamine group, a heteroarylamine group, an arylamine group, and a heterocyclic group.

In an implementation, the buffer compound may be represented by one of the following Formula 2, Formula 3, or Formula 4.

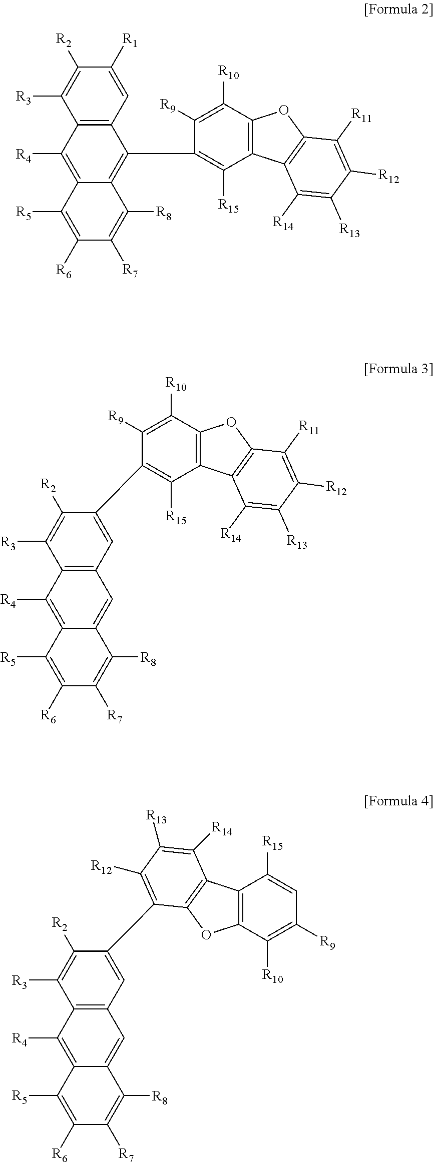

In Formulae 2-4, R₁ to R₁₈ may be defined the same as R₁ to R₁₈ of Formula 1.

In an implementation, the buffer compound may be one of the following compounds.

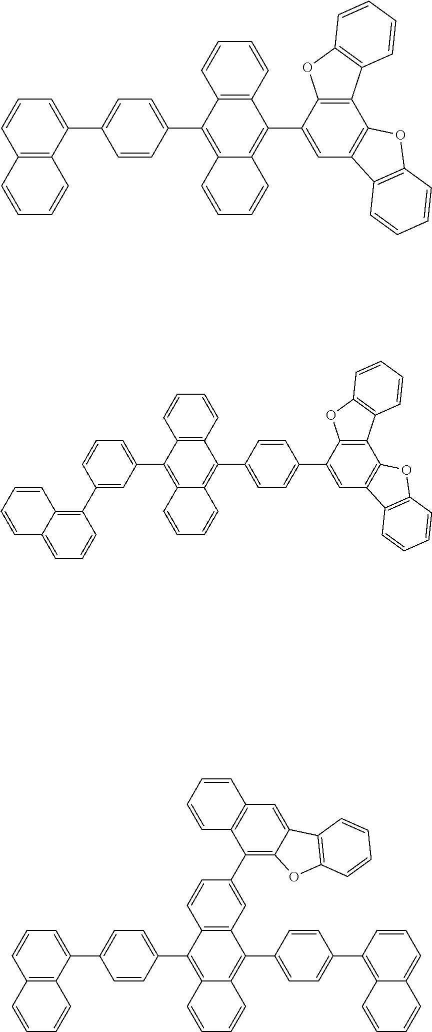 [SMILES: c1ccc2c(-c3ccc(-c4c5ccccc5c(-c5cc6c7ccccc7oc6c6c5oc5ccccc56)c5ccccc45)cc3)cccc2c1 ; c1cc(-c2cccc3ccccc23)cc(-c2c3ccccc3c(-c3ccc(-c4cc5c6ccccc6oc5c5c4oc4ccccc45)cc3)c3ccccc23)c1 ; c1ccc2c(-c3ccc(-c4c5ccccc5c(-c5ccc(-c6cccc7ccccc67)cc5)c5cc(-c6c7ccccc7cc7c6oc6ccccc67)ccc45)cc3)cccc2c1]

In an implementation, a thickness of the buffer layer BFL may be, e.g., about 10 Å to about 150 Å. Maintaining the thickness of the buffer layer BFL at about 10 Å or greater may help prevent the transfer of holes passed through the emission layer EML to the electron transport region ETR. Maintaining the thickness of the buffer layer BFL at about 150 Å or less may help smooth or facilitate the transfer of electrons from the electron transport region ETR to the emission layer EML.

In an implementation, the buffer layer BFL may further include a dopant. The dopant may be, e.g., a doped one. The dopant may include, e.g., a metal or an organic material. The metal may be, e.g., Ir, Pt, Os, Au, Cu, Re, Ru, or the like. The organic material may include, e.g., an anthracene derivative or anthracene group-containing compound.

In the case where the buffer layer BFL includes a dopant, the thickness thereof may be increased to help reduce and/or prevent the transfer of holes passed through the emission layer EML to the electron transport region ETR, when compared to that of a buffer layer BFL in which the dopant is omitted.

In the case where the buffer layer BFL includes a dopant, the thickness of the buffer layer BFL may be, e.g., about 10 Å to about 400 Å. Maintaining the thickness of the buffer layer BFL at about 10 Å or greater may help reduce and/or prevent the transfer of holes passed through the emission layer EML to the electron transport region ETR. Maintaining the thickness of the buffer layer BFL at about 400 Å or less may help smooth or facilitate the transfer of electrons from the electron transport region ETR to the emission layer EML.

The electron transport region ETR may be provided on the buffer layer BFL. The electron transport region ETR may include at least one of an electron blocking layer, an electron transport layer ETL, and an electron injection layer EIL.

The electron transport region ETR may have a single layer formed using a single material, a single layer formed using a plurality of different materials, or a multilayer structure including a plurality of layers formed using a plurality of different materials.

For example, the electron transport region ETR may have a single layer structure such as the electron injection layer EIL, and the electron transport layer ETL, or a single layer structure formed using an electron injection material and an electron transport material. In addition, the electron transport region ETR may have a single layer structure having a plurality of different materials, or a structure laminated from the anode AN of electron transport layer ETL/electron injection layer EIL, or hole blocking layer/electron transport layer ETL/electron injection layer EIL. The thickness of the electron transport region ETR may be, for example, from about 1,000 Å to about 1,500 Å.

The electron transport region ETR may be formed using various methods such as a vacuum deposition method, a spin coating method, a cast method, a Langmir-Blodgett (LB) method, an inkjet printing method, a laser printing method, and a laser induced thermal imaging (LITI) method.

When the electron transport region ETR includes the electron transport layer ETL, the electron transport region ETR may include tris(8-hydroxyquinolinato)aluminum (Alq3), 1,3,5-tri(1-phenyl-1H-benzo[d]imidazol-2-yl)phenyl (TPBi), 2,9-dimethyl-4,7-diphenyl-1,10-phenanthroline (BCP), 4,7-diphenyl-1,10-phenanthroline (Bphen), 3-(4-biphenylyl)-4-phenyl-5-tert-butylphenyl-1,2,4-triazole (TAZ), 4-(naphthalen-1-yl)-3,5-diphenyl-4H-1,2,4-triazole (NTAZ), 2-(4-biphenylyl)-5-(4-tert-butylphenyl)-1,3,4-oxadiazole (tBu-PBD), bis(2-methyl-8-quinolinolato-N1,08)-(1,1′-biphenyl-4-olato)aluminum (BAlq), berylliumbis(benzoquinolin-10-olate (Bebq2), 9,10-di(naphthalene-2-yl)anthracene (ADN), or a mixture thereof. The thickness of the electron transport layer ETL may be from about 100 Å to about 1,000 Å, e.g., may be from about 150 Å to about 500 Å. If the thickness of the electron transport layer ETL satisfies the above-described range, satisfactory electron transport property may be obtained without substantial increase of a driving voltage.

When the electron transport region ETR includes the electron injection layer EIL, the electron transport region ETR may include LiF, lithium quinolate (LiQ), Li₂O, BaO, NaCl, CsF, a lanthanide metal such as Yb, or a metal halide such as RbCl and RbI. The electron injection layer EIL also may be formed using a mixture material of a hole transport material and an insulating organo metal salt. The organo metal salt may be a material having an energy band gap of about 4 eV or more. In an implementation, the organo metal salt may include, for example, a metal acetate, a metal benzoate, a metal acetoacetate, a metal acetylacetonate, or a metal stearate. The thickness of the electron injection layer EIL may be from about 1 Å to about 100 Å, and from about 3 Å to about 90 Å. When the thickness of the electron injection layer EIL satisfies the above described range, satisfactory electron injection property may be obtained without inducing the substantial increase of a driving voltage.

The electron transport region ETR may include a hole blocking layer, as described above. The hole blocking layer may include at least one of, for example, 2,9-dimethyl-4,7-diphenyl-1,10-phenanthroline (BCP), or 4,7-diphenyl-1,10-phenanthroline (Bphen).

The cathode CAT may be provided on the electron transport region ETR. The cathode CAT may be a common electrode or a cathode. The cathode CAT may be a transmissive electrode, a transflective electrode or a reflective electrode. When the cathode CAT is the transmissive electrode, the cathode CAT may include a transparent metal oxide, for example, ITO, IZO, ZnO, ITZO, etc.

When the cathode CAT is the transflective electrode or the reflective electrode, the cathode CAT may include Ag, Mg, Cu, Al, Pt, Pd, Au, Ni, Nd, Ir, Cr, Li, Ca, LiF/Ca, LiF/Al, Mo, Ti, a compound thereof, or a mixture thereof (for example, a mixture of Ag and Mg). The cathode CAT may have a multilayered structure including a reflective layer or a transflective layer formed using the above-described materials and a transparent conductive layer formed using ITO, IZO, ZnO, ITZO, etc.

In an implementation, the cathode CAT may be connected with an auxiliary electrode. If the cathode CAT is connected with the auxiliary electrode, the resistance of the cathode CAT may decrease.

In the organic light emitting device OEL, according to the application of voltages to each of the anode AN and the cathode CAT, holes injected from the anode AN may transfer via the hole transport region HTR to the emission layer EML, and electrons injected from the cathode CAT may transfer via the electron transport region ETR to the emission layer EML. The electrons and the holes are recombined in the emission layer EML to generate excitons, and the excitons may emit light via transition from an excited state to a ground state.

When the organic light emitting device OEL is a top emission type, the anode AN may be a reflective electrode, and the cathode CAT may be a transmissive electrode or a transflective electrode. When the organic light emitting device OEL is a bottom emission type, the anode AN may be a transmissive electrode or a transflective electrode, and the cathode CAT may be a reflective electrode.

The organic light emitting device according to an embodiment may include a buffer layer including a buffer compound represented by Formula 1, and may help increase luminous efficiency at low grey scale and improve luminous efficiency at low grey scale. The low grey scale may mean 0 to 80 grey levels.

Hereinafter a method of manufacturing a display device according to an embodiment will be explained. The explanation will be concentrated on different points from the organic light emitting device according to an embodiment described above, and unexplained parts will follow the explanation on the organic light emitting device according to an embodiment described above.

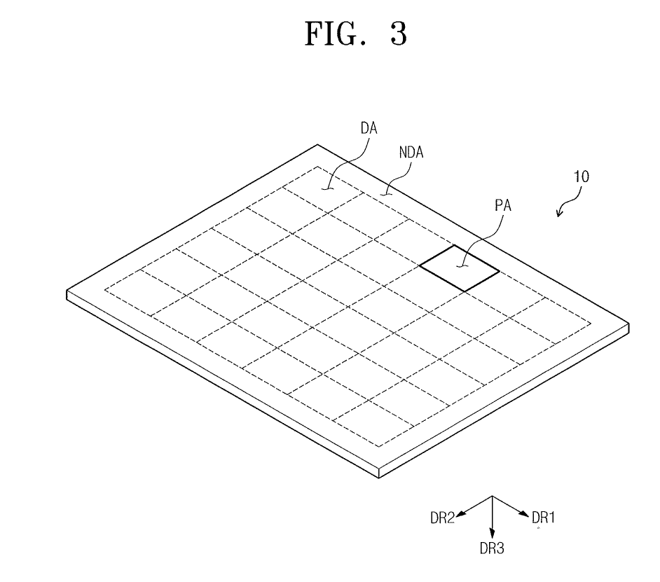

FIG. 3 illustrates a perspective view schematically showing a display device according to an embodiment.

Referring to FIG. 3, a display device 10 according to an embodiment may be divided into a display area DA and a non-display area NDA. The display area DA may display images. When seen from the direction of the thickness of the display device 10 (for example, in DR3), the display area DA may have approximately a rectangle shape.

The display area DA may include a plurality of pixel areas PA. The pixel areas PA may be disposed in a matrix shape. In the pixel areas PA, the plurality of pixels PX may be disposed. Each of the pixels PX may include sub-pixels. Each of the pixels PX may include an organic light emitting device (OEL in FIG. 1).

A non-display area NDA may not display images. When seen from the direction of the thickness of the display device 10 (in DR3), the non-display area NDA may, for example, surround the display area DA. The non-display area NDA may be adjacent to the display area DA in a first direction DR1 and a second direction DR2.

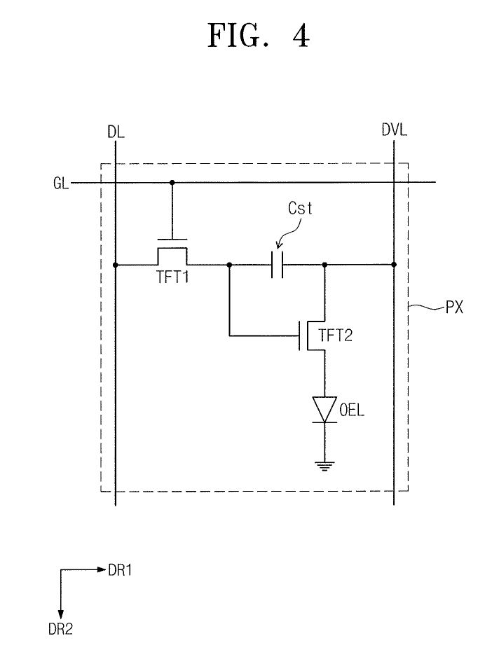

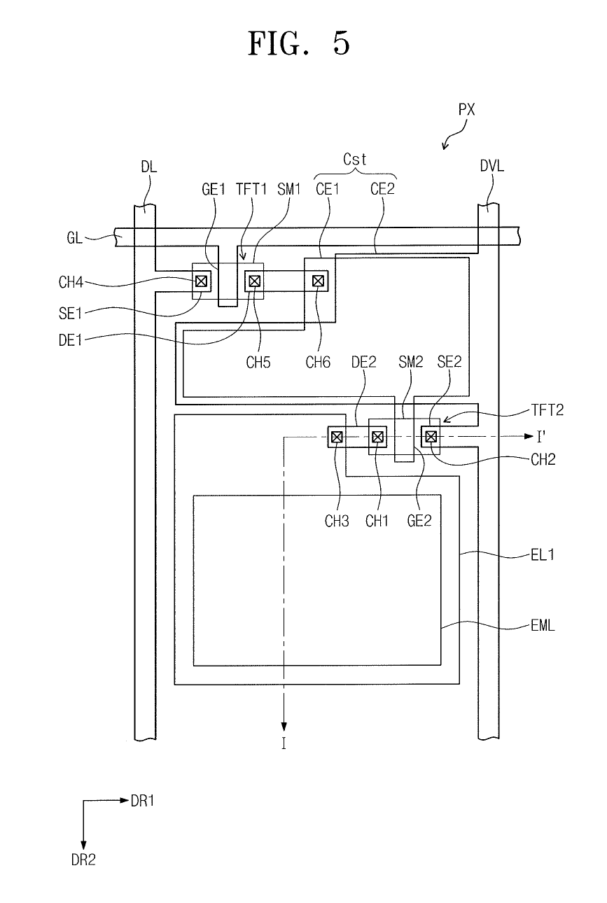

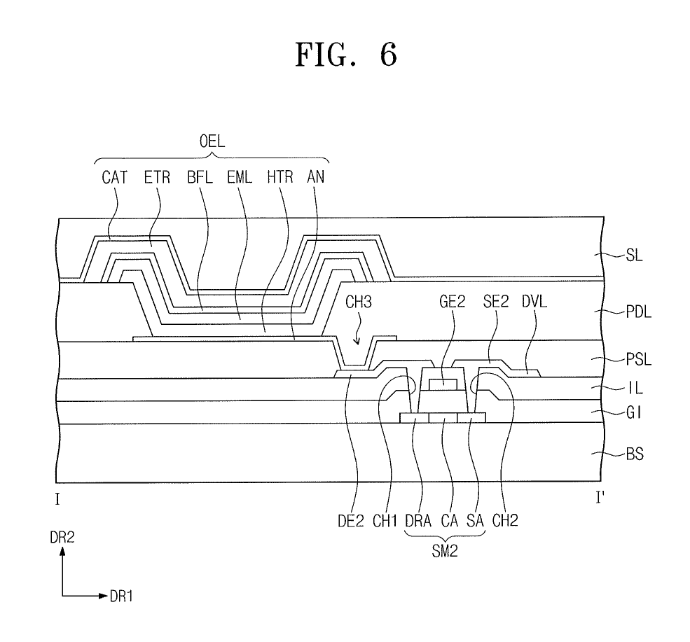

FIG. 4 illustrates a circuit diagram of a pixel included in a display device according to an embodiment. FIG. 5 illustrates a plan view of a pixel included in a display device according to an embodiment. FIG. 6 illustrates a schematic cross-sectional view taken along line I-I′ in FIG. 5.

Referring to FIGS. 1 to 6, each of the pixels PX may include a wire part including a gate line GL, a data line DL, and a driving voltage line DVL. Each of the pixels PX may include thin film transistors TFT1 and TFT2 connected to the wire part, an organic light emitting device OEL connected to the thin film transistors TFT1 and TFT2, and a capacitor Cst. Each of the pixels PX may emit light having a specific color, for example, one of red light, green light, blue light, white light, yellow light, or cyan light.

From the plan view of FIG. 4, each of the pixels PX have a rectangular shape, however each of the pixels PX may have at least one shape of a circle, an ellipse, a square, a parallelogram, a trapezoid, or a rhombus. In an implementation, each of the pixels PX may have, for example, a quadrangle having at least one rounded corner from the plan view.

The gate line GL may be extended in a first direction DR1. The data line DL may be extended in a second direction DR2 crossing the gate line GL. The driving voltage line DVL may be extended in substantially the same direction as the data line DL, that is, the second direction DR2. The gate line GL transmits scanning signals to the thin film transistors TFT1 and TFT2, and the data line DL transmits data signals to the thin film transistors TFT1 and TFT2, and the driving voltage line DVL provides driving voltages to the thin film transistors TFT1 and TFT2.

The thin film transistors TFT1 and TFT2 may include a driving thin film transistor TFT2 for controlling the organic light emitting device OEL, and a switching thin film transistor TFT1 for switching the driving thin film transistor TFT2. In an embodiment, each of the pixels PX includes two thin film transistors TFT1 and TFT2. Each of the pixels PX may include one thin film transistor and one capacitor, or each of the pixels PX may include at least three thin film transistors and at least two capacitors.

The switching thin film transistor TFT1 may include a first gate electrode GE1, a first source electrode SE1, and a first drain electrode DE1. The first gate electrode GE1 may be connected to the gate line GL, and the first source electrode SE1 may be connected to the data line DL. The first drain electrode DE1 may be connected to a first common electrode CE1 via a fifth contact hole CH5. The switching thin film transistor TFT1 may transmit data signals applied to the data line DL to the driving thin film transistor TFT2 according to scanning signals applied to the gate line GL.

The driving thin film transistor TFT2 may include a second gate electrode GE2, a second source electrode SE2, and a second drain electrode DE2. The second gate electrode GE2 may be connected to the first common electrode CE1. The second source electrode SE2 may be connected to the driving voltage line DVL. The second drain electrode DE2 may be connected to the anode AN via a third contact hole CH3.

The capacitor Cst may be connected between the second gate electrode GE2 and the second source electrode SE2 of the driving thin film transistor TFT2, and charge and maintain data signals inputted to the second gate electrode GE2 of the driving thin film transistor TFT2. The capacitor Cst may include the first common electrode CE1 connected to the first drain electrode DE1 via a sixth contact hole CH6 and a second common electrode CE2 connected to the driving voltage line DVL.

The display device 10 according to an embodiment may include a base substrate BS on which thin film transistors TFT1 and TFT2, and an organic light emitting device OEL are laminated. A suitable substrate may be used as the base substrate BS, and may be formed using an insulating material such as glass, plastics, and quartz. As an organic polymer forming the base substrate BS, polyethylene terephthalate (PET), polyethylene naphthalate (PEN), polyimide, polyethersulfone, etc. may be used. The base substrate BS may be selected in consideration of mechanical strength, thermal stability, transparency, surface smoothness, easiness of handling, water-proof properties, etc.

On the base substrate BS, a substrate buffer layer may be provided. The substrate buffer layer may prevent the diffusion of impurities into the switching thin film transistor TFT1 and the driving thin film transistor TFT2. The substrate buffer layer may be formed using silicon nitride (SiNx), silicon oxide (SiOx), silicon oxynitride (SiOxNy), etc., and may be omitted according to the material of the base substrate BS and process conditions.

On the base substrate BS, a first semiconductor layer SM1 and a second semiconductor layer SM2 may be provided. The first semiconductor layer SM1 and the second semiconductor layer SM2 may be formed using a semiconductor material and function as active layers of the switching thin film transistor TFT1 and the driving thin film transistor TFT2, respectively. Each of the first semiconductor layer SM1 and the second semiconductor layer SM2 may include a source area SA, a drain area DRA, and a channel area CA provided between the source area SA and the drain area DRA. Each of the first semiconductor layer SM1 and the second semiconductor layer SM2 may be formed by selecting inorganic semiconductor or organic semiconductor, respectively. The source area SA and the drain area DRA may be doped with n-type impurities or p-type impurities.

On the first semiconductor layer SM1 and the second semiconductor layer SM2, a gate insulating layer GI may be provided. The gate insulating layer GI may cover the first semiconductor layer SM1 and the second semiconductor layer SM2. The gate insulating layer GI may include at least one of an organic insulating material or an inorganic insulating material.

On the gate insulating layer GI, a first gate electrode GE1 and a second gate electrode GE2 may be provided. Each of the first gate electrode GE1 and the second gate electrode GE2 may be formed to cover corresponding areas in the channel area CA of the first semiconductor layer SM1 and the second semiconductor layer SM2.

On the insulating interlayer IL, a first source electrode SE1, a first drain electrode DE1, a second source electrode SE2, and a second drain electrode DE2 may be provided. The second drain electrode DE2 may make contact with the drain area DRA of the second semiconductor layer SM2 via a first contact hole CH1 formed in the gate insulating layer GI and the insulating interlayer IL, and the second source electrode SE2 may make contact with the source area SA of the second semiconductor layer SM2 by a second contact hole CH2 formed in the gate insulating layer GI and the insulating interlayer IL. The first source electrode SE1 may make contact with a source area (not shown) of the first semiconductor layer SM1 via a fourth contact hole CH4 formed in the gate insulating layer GI and the insulating interlayer IL, and the first drain electrode DE1 may make contact with a drain area (not shown) of the first semiconductor layer SM1 via a fifth contact hole CH5 formed in the gate insulating layer GI and the insulating interlayer IL.

On the first source electrode SE1, the first drain electrode DE1, the second source electrode SE2, and the second drain electrode DE2, a passivation layer PSL may be provided. The passivation layer PSL may play the role of passivating the switching thin film transistor TFT1 and the driving thin film transistor TFT2, or the role of planarizing the top surface thereof.

On the passivation layer PSL, an anode AN may be provided. The anode AN may be, for example, a pixel electrode or an anode. The anode AN may be connected to the second drain electrode DE2 of the driving thin film transistor TFT2 via the third contact hole CH3 formed in the passivation layer PSL.

The hole transport region HTR may be provided on the anode AN. The hole transport region HTR may include at least one of a hole injection layer HIL, a hole transport layer HTL, a buffer layer, or an electron blocking layer.

The emission layer EML may be provided on the hole transport region HTR. The emission layer EML may have a single layer formed using a single material, a single layer formed using a plurality of different materials, or a multilayer structure having a plurality of layers formed using a plurality of different materials.

The buffer layer BFL may be provided on the emission layer EML. The buffer layer BFL may include a buffer compound represented by the following Formula 1.

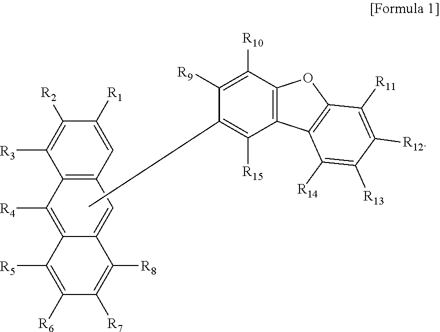

In Formula 1, R₁ to R₁₈ may each independently be or include, e.g., hydrogen, deuterium, a substituted or unsubstituted aromatic group, or a substituted or unsubstituted heteroaromatic group. In an implementation, adjacent ones of R₁ to R₁₈ may be separate or may be fused to form substituted or unsubstituted condensed aromatic groups or substituted or unsubstituted condensed heteroaromatic groups.

In an implementation, in Formula 1, R₁ to R₁₈ may each independently be, e.g., a halogen group, a nitrile group, a nitro group, an amino group, a phosphine oxide group, an alkoxy group, an aryloxy group, an alkylthioxy group, an arylthioxy group, an alkylsulfoxy group, an arylsulfoxy group, a silyl group, a boron group, an alkyl group, a cycloalkyl group, an alkenyl group, an aryl group, an aralkyl group, an aralkenyl group, an alkylaryl group, an alkylamine group, a heteroarylamine group, an arylamine group, or a heterocyclic group.

In an implementation, the buffer compound may be represented by one of the following Formula 2, Formula 3, or Formula 4.

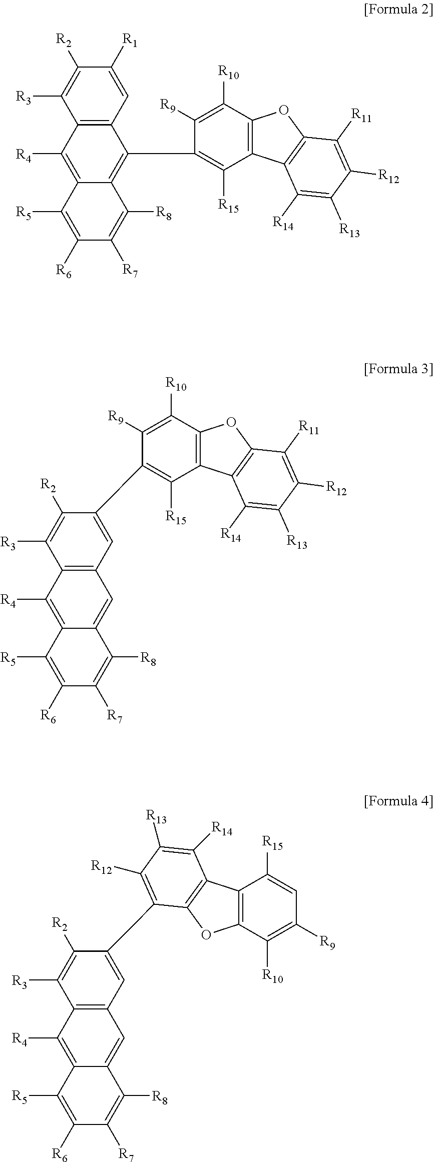

In Formulae 2-4, R₁ to R₁₈ may be defined the same as R₁ to R₁₈ of Formula 1.

In an implementation, the buffer compound may be one of the following compounds.

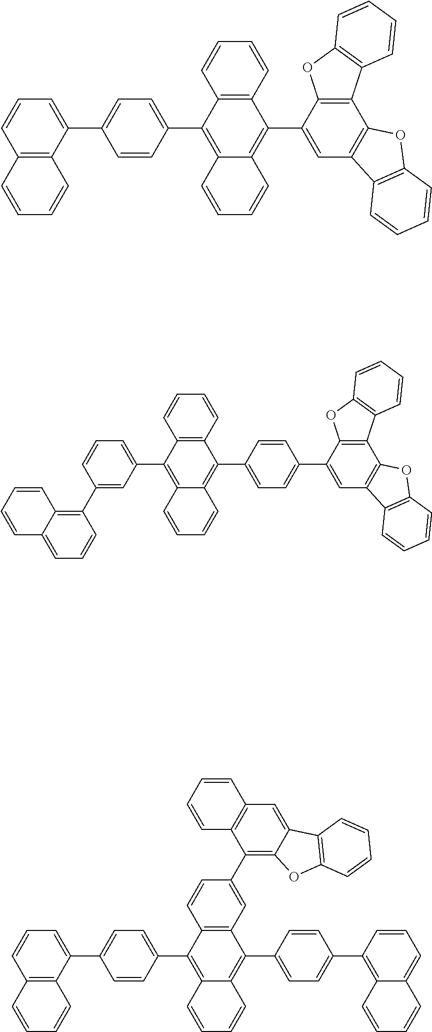 [SMILES: c1ccc2c(-c3ccc(-c4c5ccccc5c(-c5cc6c7ccccc7oc6c6c5oc5ccccc56)c5ccccc45)cc3)cccc2c1 ; c1cc(-c2cccc3ccccc23)cc(-c2c3ccccc3c(-c3ccc(-c4cc5c6ccccc6oc5c5c4oc4ccccc45)cc3)c3ccccc23)c1 ; c1ccc2c(-c3ccc(-c4c5ccccc5c(-c5ccc(-c6cccc7ccccc67)cc5)c5cc(-c6c7ccccc7cc7c6oc6ccccc67)ccc45)cc3)cccc2c1]

In an implementation, a thickness of the buffer layer BFL may be, e.g., about 10 Å to about 150 Å. Maintaining the thickness of the buffer layer BFL at about 10 Å or greater may help prevent the transfer of holes passed through the emission layer EML to the electron transport region ETR. Maintaining the thickness of the buffer layer BFL at about 150 Å or less may help smooth or facilitate the transfer of electrons from the electron transport region ETR to the emission layer EML.

In an implementation, the buffer layer BFL may further include a dopant. The dopant may be, e.g., a doped one. The dopant may include, e.g., a metal or an organic material. The metal may be, e.g., Ir, Pt, Os, Au, Cu, Re, Ru, or the like. The organic material may include, e.g., an anthracene derivative or anthracene group-containing compound.

In the case where the buffer layer BFL includes a dopant, the thickness thereof may be increased to help reduce and/or prevent the transfer of holes passed through the emission layer EML to the electron transport region ETR, when compared to that of a buffer layer BFL in which the dopant is omitted.

In the case where the buffer layer BFL includes a dopant, the thickness of the buffer layer BFL may be, e.g., about 10 Å to about 400 Å. Maintaining the thickness of the buffer layer BFL at about 10 Å or greater may help reduce and/or prevent the transfer of holes passed through the emission layer EML to the electron transport region ETR. Maintaining the thickness of the buffer layer BFL at about 400 Å or less may help smooth or facilitate the transfer of electrons from the electron transport region ETR to the emission layer EML.

The electron transport region ETR may be provided on the buffer layer BFL. The electron transport region ETR may include at least one of an electron blocking layer, an electron transport layer ETL, and an electron injection layer ETL.

A cathode CAT may be provided on the electron transport region ETR. The cathode CAT may be a common electrode or a cathode. In an implementation, the cathode CAT may be connected to an auxiliary electrode.

On the cathode CAT, a sealing layer SL may be provided. The sealing layer SL may cover the cathode CAT. The sealing layer SL may include at least one layer of an organic layer, an inorganic layer, and a hybrid layer including both an organic material and an inorganic material. The sealing layer SL may be a single layer, or a multilayer. The sealing layer SL may be, for example, a thin film sealing layer. The sealing layer SL may passivate the organic light emitting device OEL.

The display device according to an embodiment may include the buffer layer including the buffer compound represented by Formula 1, and may help increase luminous efficiency at low grey scale and may help improve the deterioration of the luminous efficiency of the display device at low grey scale.

### EXAMPLES

The following Examples and Comparative Examples are provided in order to highlight characteristics of one or more embodiments, but it will be understood that the Examples and Comparative Examples are not to be construed as limiting the scope of the embodiments, nor are the Comparative Examples to be construed as being outside the scope of the embodiments. Further, it will be understood that the embodiments are not limited to the particular details described in the Examples and Comparative Examples.

### Example 1

An anode was formed using ITO on a glass substrate, a hole injection layer was formed using 2-TNATA, a hole transport layer was formed using N,N′-bis(3-methylphenyl)-N,N′-diphenyl0[1,1-biphenyl]-4,4′-diamine (TPD), an emission layer was formed using 9,10-di(2-naphthyl)anthracene (ADN) doped with 2,5,8,11-tetra-t-butylperylene (TBP), a buffer layer was formed using the following Compound 1 to have a thickness of about 10 Å, an electron transport layer was formed using Alq3, an electron injection layer was formed using LiF, and a cathode was formed using Al.

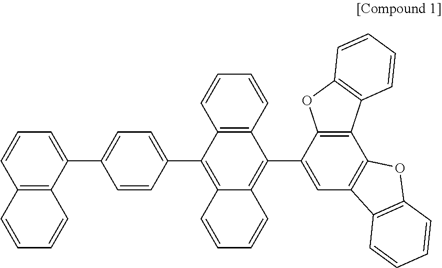 [SMILES: c1ccc2c(-c3ccc(-c4c5ccccc5c(-c5cc6c7ccccc7oc6c6c5oc5ccccc56)c5ccccc45)cc3)cccc2c1]

### Example 2

The same procedure was conducted as described in Example 1 except for forming the buffer layer to a thickness of about 30 Å.

### Example 3

The same procedure was conducted as described in Example 1 except for forming the buffer layer to a thickness of about 50 Å.

### Example 4

The same procedure was conducted as described in Example 1 except for forming the buffer layer using an Ir dopant and Compound 1 to a thickness of about 30 Å.

### Example 5

The same procedure was conducted as described in Example 4 except for forming the buffer layer to a thickness of about 100 Å.

### Example 6

The same procedure was conducted as described in Example 4 except for forming the buffer layer to a thickness of about 150 Å.

### Comparative Example

The same procedure was conducted as described in Example 4 except for omitting forming a buffer layer.

Experimental Results

Luminous efficiency was measured for Examples 1 to 6 and the Comparative Example. The luminous efficiency of the organic light emitting devices was measured while driving under current density conditions of 10 mA/cm².

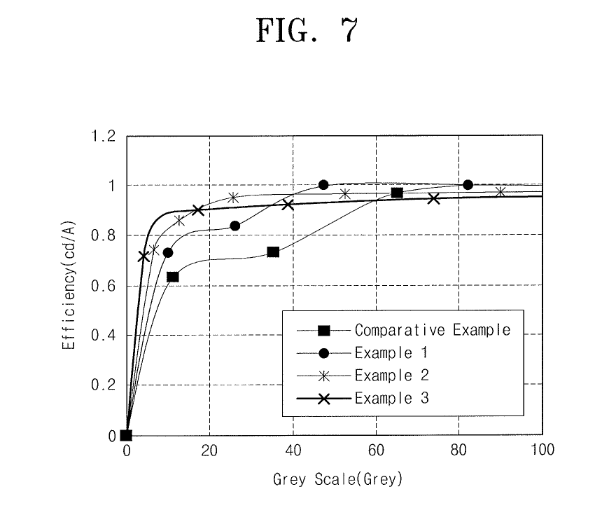

Referring to FIG. 7, it may be seen that the luminous efficiency was decreased for the Comparative Example at low grey scale with a grey level from 0 to 80. However, the luminous efficiency was improved at low grey scale with the grey level from 0 to 80 for Examples 1 to 3, when compared to that of the Comparative Example.

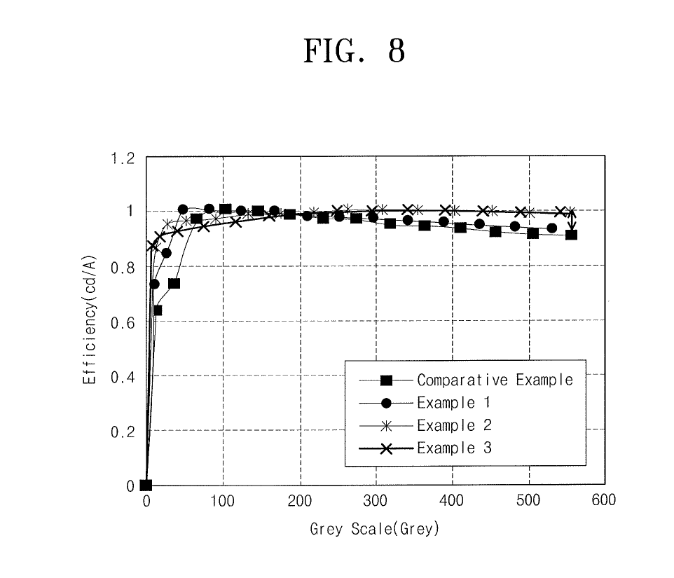

Referring to FIG. 8, it may be seen that the luminous efficiency was higher at low grey scale with the grey level of 300 or more for Examples 1 to 3, when compared to that of the Comparative Example.

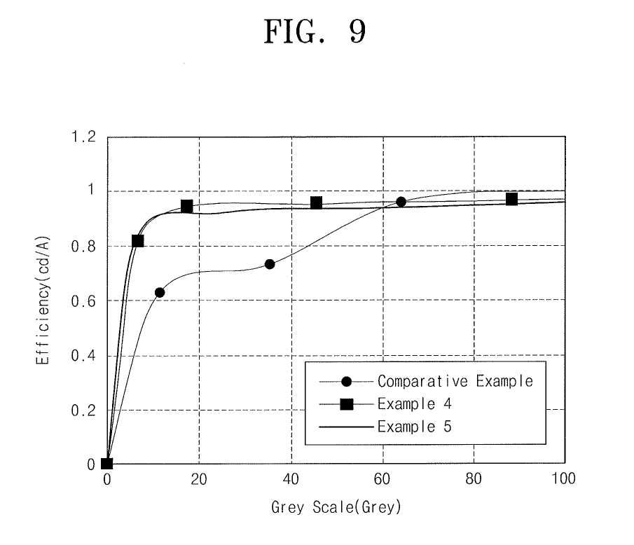

Referring to FIG. 9, it may be seen that the luminous efficiency was decreased at low grey scale with the grey level from 0 to 80 for the Comparative Example. However, the luminous efficiency was improved at low grey scale with the grey level from 0 to 80 for Examples 4 and 5, when compared to that of the Comparative Example.

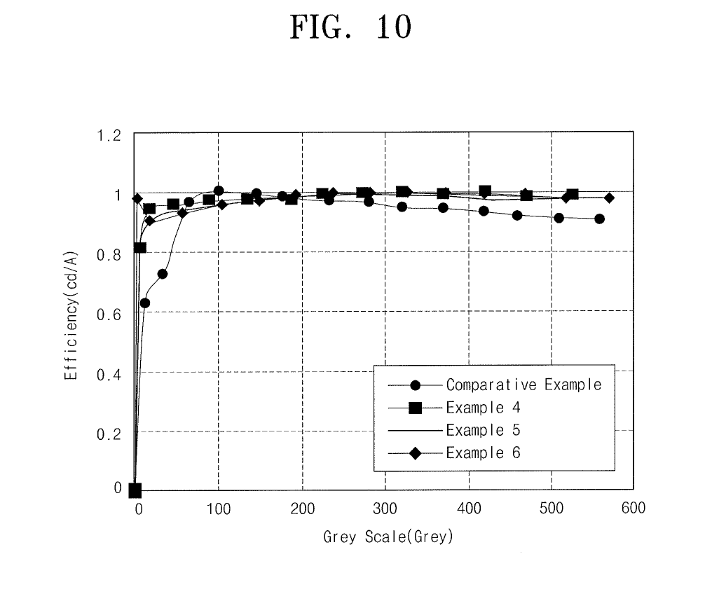

Referring to FIG. 10, it may be seen that the luminous efficiency was higher at low grey scale with the grey level of 300 or more for Examples 4 to 6, when compared to that of the Comparative Example.

The embodiments may provide an organic light emitting device capable of increasing luminous efficiency at low grey scale and capable of improving luminous efficiency at low grey scale.

In the organic light emitting device according to an embodiment, luminous efficiency may be increased, and luminous efficiency may be improved at low grey scale.

In a display device according to an embodiment, luminous efficiency may be increased, and luminous efficiency may be improved at low grey scale.

Example embodiments have been disclosed herein, and although specific terms are employed, they are used and are to be interpreted in a generic and descriptive sense only and not for purpose of limitation. In some instances, as would be apparent to one of ordinary skill in the art as of the filing of the present application, features, characteristics, and/or elements described in connection with a particular embodiment may be used singly or in combination with features, characteristics, and/or elements described in connection with other embodiments unless otherwise specifically indicated. Accordingly, it will be understood by those of skill in the art that various changes in form and details may be made without departing from the spirit and scope of the present invention as set forth in the following claims.

What is claimed is:

1. An organic light emitting device, comprising:
an anode;
a hole transport region on the anode;
an emission layer on the hole transport region;
a buffer layer on the emission layer;
an electron transport region on the buffer layer; and
a cathode on the electron transport region,
wherein the buffer layer includes a buffer compound represented by the following Formula 1: 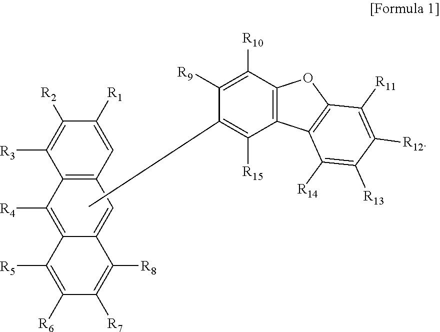
wherein, in Formula 1, R₁ to R₁₈ are each independently hydrogen, deuterium, a substituted or unsubstituted aromatic group, or a substituted or unsubstituted heteroaromatic group, adjacent ones of R₁ to R₁₈ being separate or fused to form substituted or unsubstituted condensed aromatic groups or substituted or unsubstituted condensed heteroaromatic groups.

2. The organic light emitting device as claimed in claim 1, wherein the buffer compound is represented by one of the following Formula 2, Formula 3, or Formula 4: 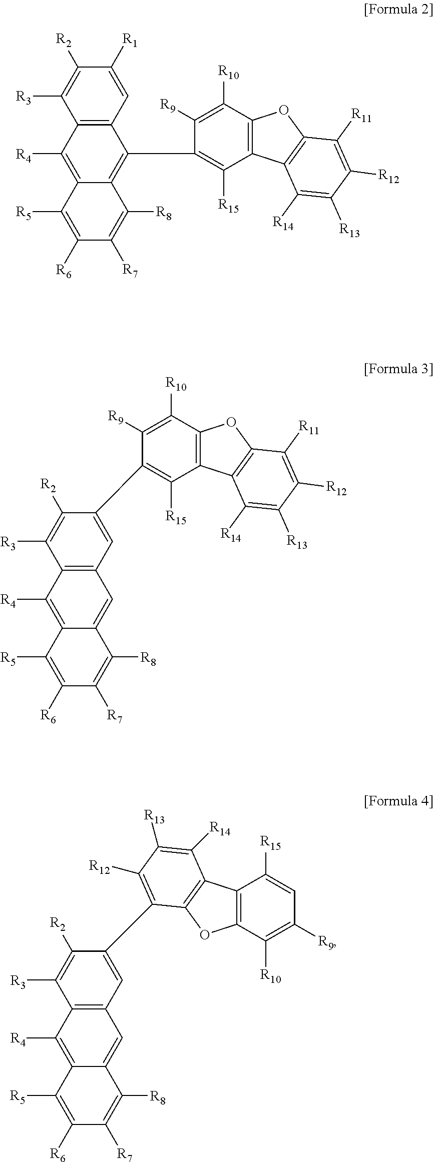
wherein, in Formulae 2, 3, and 4, R₁ to R₁₈ are defined the same as R₁ to R₁₈ of Formula 1.

3. The organic light emitting device as claimed in claim 1, wherein the buffer compound is one of the following compounds:

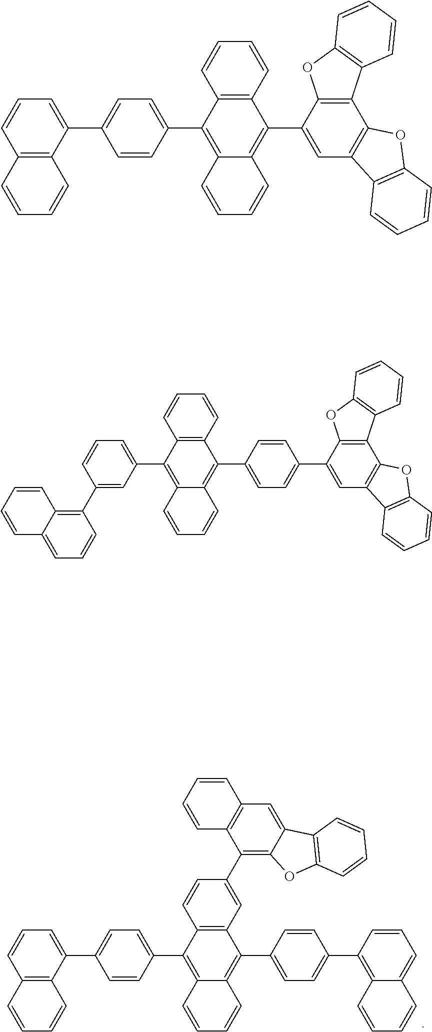 [SMILES: c1ccc2c(-c3ccc(-c4c5ccccc5c(-c5cc6c7ccccc7oc6c6c5oc5ccccc56)c5ccccc45)cc3)cccc2c1 ; c1cc(-c2cccc3ccccc23)cc(-c2c3ccccc3c(-c3ccc(-c4cc5c6ccccc6oc5c5c4oc4ccccc45)cc3)c3ccccc23)c1 ; c1ccc2c(-c3ccc(-c4c5ccccc5c(-c5ccc(-c6cccc7ccccc67)cc5)c5cc(-c6c7ccccc7cc7c6oc6ccccc67)ccc45)cc3)cccc2c1]

4. The organic light emitting device as claimed in claim 1, wherein a thickness of the buffer layer is about 10 Å to about 150 Å.

5. The organic light emitting device as claimed in claim 1, wherein the buffer layer further includes a dopant.

6. The organic light emitting device as claimed in claim 5, wherein the dopant includes Ir, Pt, Os, Au, Cu, Re, Ru, or an anthracene group-containing compound.

7. The organic light emitting device as claimed in claim 5, wherein a thickness of the buffer layer is about 10 Å to about 400 Å.

8. The organic light emitting device as claimed in claim 1, wherein the hole transport region includes:
a hole injection layer; and
a hole transport layer on the hole injection layer.

9. The organic light emitting device as claimed in claim 1, wherein the electron transport region includes:
an electron transport layer; and
an electron injection layer on the electron transport layer.

10. A display device comprising a plurality of pixels, wherein at least one of the pixels includes:
an anode;
a hole transport region on the anode;
an emission layer on the hole transport region;
a buffer layer on the emission layer;
an electron transport region on the buffer layer; and
a cathode on the electron transport region,
wherein the buffer layer includes a buffer compound represented by the following Formula 1: 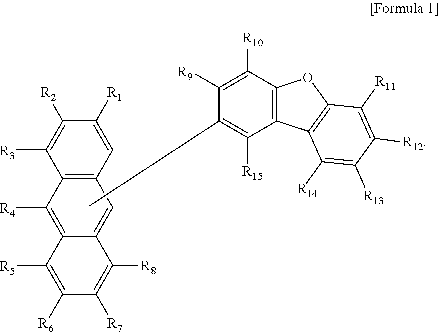
wherein, in Formula 1, R₁ to R₁₈ are each independently hydrogen, deuterium, a substituted or unsubstituted aromatic group, or a substituted or unsubstituted heteroaromatic group, adjacent ones of R₁ to R₁₈ being separate or fused to form substituted or unsubstituted condensed aromatic groups or substituted or unsubstituted condensed heteroaromatic groups.

11. The display device as claimed in claim 10, wherein the buffer compound is represented by one of the following Formula 2, Formula 3, or Formula 4: 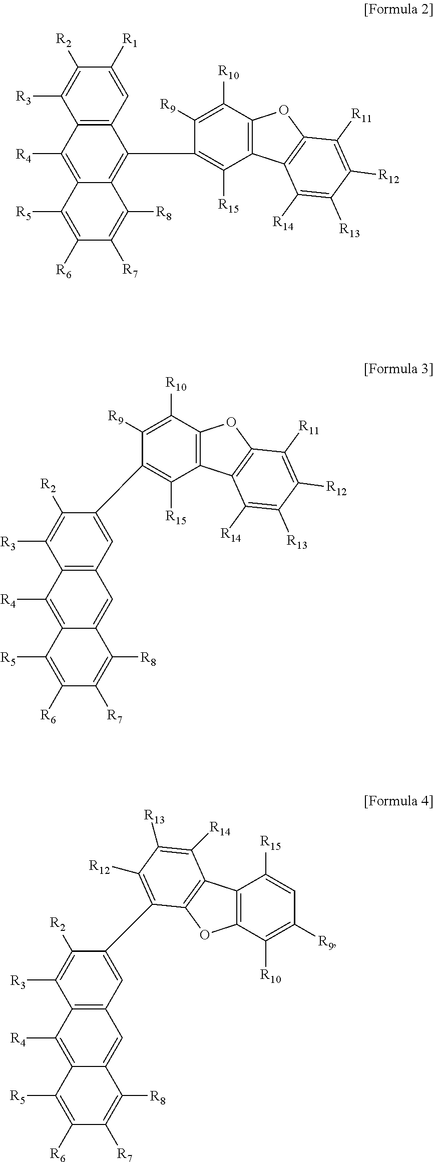
wherein, in Formulae 2, 3, and 4, R₁ to R₁₈ are defined the same as R₁ to R₁₈ of Formula 1

12. The display device as claimed in claim 10, wherein the buffer compound is one of the following compounds:

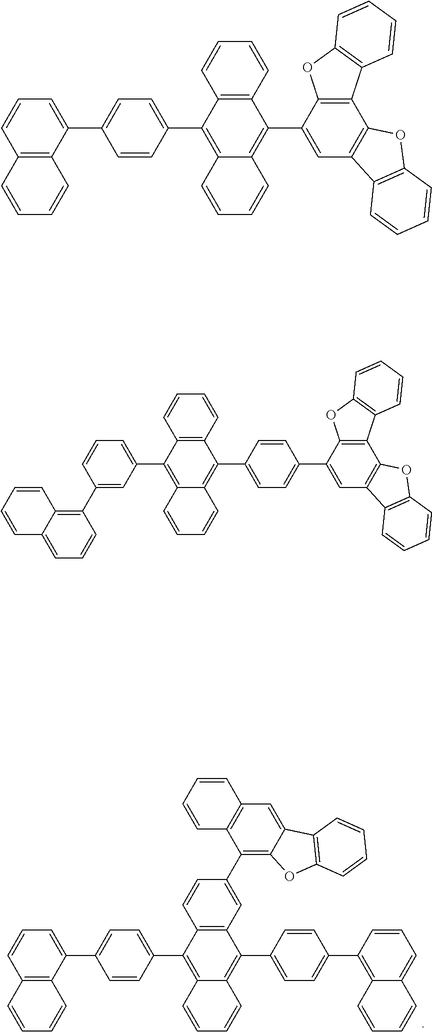 [SMILES: c1ccc2c(-c3ccc(-c4c5ccccc5c(-c5cc6c7ccccc7oc6c6c5oc5ccccc56)c5ccccc45)cc3)cccc2c1 ; c1cc(-c2cccc3ccccc23)cc(-c2c3ccccc3c(-c3ccc(-c4cc5c6ccccc6oc5c5c4oc4ccccc45)cc3)c3ccccc23)c1 ; c1ccc2c(-c3ccc(-c4c5ccccc5c(-c5ccc(-c6cccc7ccccc67)cc5)c5cc(-c6c7ccccc7cc7c6oc6ccccc67)ccc45)cc3)cccc2c1]

13. The display device as claimed in claim 10, wherein a thickness of the buffer layer is about 10 Å to about 150 Å.

14. The display device as claimed in claim 10, wherein the buffer layer further includes a dopant.

15. The display device as claimed in claim 14, wherein the dopant includes Ir, Pt, Os, Au, Cu, Re, Ru, or an anthracene group-containing compound.

16. The display device as claimed in claim 14, wherein a thickness of the buffer layer is about 10 Å to about 400 Å.
